(19) United States
(12) Patent Application Publication
(10) Pub. No.: US 2006/0116529 A1
(43) Pub. Date: Jun. 1, 2006

(54) Method for the production of isocyanates

(75) Inventors: Andreas Wolfert, BAD RAPPENAU (DE); Hans-Jurgen Pallasch, Kallstadt (DE); Eckhard Stroefer, Mannheim (DE); Ulrich Penzel, Tettau (DE); Filip Deberdt, Muizen (BE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF AKTIENGESELLSCHAFT, LUDWIGSHAFEN (DE)

(21) Appl. No.: 10/538,474

(22) PCT Filed: Dec. 16, 2003
(86) PCT No.: PCT/EP03/14290

§ 371 Date: Jun. 7, 2005

(30) Foreign Application Priority Data
Dec. 20, 2002 (DE) 102 61 187.4

(51) Int. Cl. C07C 263/10

(52) U.S. Cl. 560/347

(57) ABSTRACT

The invention provides a process for preparing isocyanates by reacting amines with phosgene, wherein the phosgene-containing feed stream has a hydrogen chloride content of more than 0.8% by mass.

The present invention relates to a process for preparing isocyanates by reacting amines with phosgene, wherein the phosgene-containing feed stream has a content of hydrogen chloride (hereinafter referred to as HCl) of more than 0.8% by mass.

Various processes for preparing isocyanates by reacting amines with phosgene have already been described in the literature.

U.S. Pat. No. 3,234,253 describes a continuous two-stage process in which amine and phosgene are mixed in the first stage and HCl and phosgene are subsequently introduced in the second, hot phosgenation stage to increase the yield. This process has the disadvantage of low industrially achievable yields.

WO 96/16028 describes a continuous process for preparing isocyanates, in which the reaction is carried out in one temperature stage and isocyanate is used as solvent for the phosgene, with the chlorine content of the isocyanate being less than 2%. A tube reactor can be used for the phosgenation. A disadvantage of the process is that the isocyanate is continuously recirculated to the reaction zone where it can react in the presence of the free amine to form ureas which are precipitated as solids. Stable operation of such a process is put at risk by solids problems. The large quantity of circulated isocyanate results in a relatively large reaction volume, which is associated with an undesirably high outlay in terms of apparatus.

U.S. Pat. No. 4,581,174 describes the continuous preparation of organic monoisocyanates and/or polyisocyanates by phosgenation of the primary amine in a mixing circuit with partial recirculation of the isocyanate-containing reaction mixture, with the HCl content of the recirculated mixture being less than 0.5%. Here too, the continuous recirculation of the isocyanate to the reaction zone promotes urea formation by reaction with free amine. The precipitated urea puts stable operation of the process at risk.

GB 737 442 describes the recovery of phosgene from the synthesis of isocyanates. The recovered phosgene has an HCl content of from 0.5 to 0.7%.

EP 322 647 describes the continuous preparation of monoisocyanates or polyisocyanates by use of a nozzle having an annular orifice. A good yield is achieved in the process due to good mixing of amine and phosgene. A disadvantage is the tendency of the amine feed holes to become blocked.

It is known that good mixing contributes to improvement of the yield. There have therefore been many attempts, as described in EP 322 647, to improve the yield by improving mixing. An improvement in mixing is usually achieved by increasing the flow velocities. At a volume flow through the mixing apparatus determined by the stoichiometry of the process, this is achieved by reducing the size of the inlet openings and throughput cross sections for the incoming streams. However, the smaller the inlet openings and throughput cross sections into the mixing apparatus, the higher the risk of blockages occurring.

It is also known that the use of a high excess of phosgene over the amine used leads to high selectivities to the isocyanate to be prepared and thus has a decisive influence on the economics of the production process. As the ratio of phosgene to amino groups increases, the phosgene holdup in the plant and the plant volume also increase. However, due to the toxicity of phosgene, a very low phosgene holdup and a compact plant construction are desired. This at the same time reduces the capital costs of the plant and thus improves the economics of the process.

It is an object of the present invention to provide a process for preparing isocyanates which allows the resulting reactions to be carried out with high selectivity and high space-time yield and high operating stability, so that the process can be carried out economically in a physically compact plant.

In particular, it is an object of the invention to provide a process for preparing isocyanates which makes it possible to achieve an improvement in the yield compared to the processes which have been described hitherto. It is an object of the invention to achieve an improvement in the yield independently of the improvement in mixing.

We have found that an improvement in the yield of the process can be achieved when the phosgene solution used for mixing with the amine solution has an HCl content of more than 0.8% by mass. In particular, the extent of urea formation during the phosgenation was able to be reduced by means of an HCl content of more than 0.8% by mass based on the mixture of phosgene and HCl prior to mixing of amine solution and phosgene or phosgene solution.

The technical effect of the process of the present invention is surprising because HCl is formed in considerable amounts during the reaction of isocyanate formation. In the reaction, the phosgene reacts firstly with the amino groups to eliminate hydrogen chloride and form the carbamoyl chloride. The carbamoyl chloride group is then converted into an isocyanate group with further elimination of hydrogen chloride.

The present invention accordingly provides a process for preparing isocyanates by reacting amines with phosgene, wherein the phosgene-containing feed stream has a hydrogen chloride content of more than 0.8% by mass.

The invention further provides for the use of phosgene having a hydrogen chloride content of more than 0.8% by mass for preparing isocyanates by phosgenation of primary amines.

Finally, the invention provides a production plant for preparing isocyanates by reacting primary amines with phosgene, comprising an amine reservoir, a phosgene reservoir, a mixing apparatus, a reactor and a work-up apparatus, wherein the phosgene-containing feed stream fed into the mixing apparatus from the phosgene reservoir has a hydrogen chloride content of more than 0.8% by mass.

According to the present invention, it is necessary for the phosgene required for the reaction and fed in (=phosgene-containing feed stream fed in) to have a hydrogen chloride content of more than 0.8% by mass. The phosgene-containing feed stream preferably has a hydrogen chloride content of from 1.3% by mass to 15% by mass, more preferably from 1.7% by mass to <10% by mass, particularly preferably from 2 to <7% by mass. Here, the percentages by mass are based on the sum of the phosgene stream and the HCl streams. This reference stream expressly does not include the mass of the solvent if one or more solvents are additionally present in the phosgene-containing stream fed to the reaction or mixing apparatus.

Furthermore, it is preferred that the phosgene stream fed to mixing of amine and phosgene streams already contains the abovementioned amount of HCl. The amount of HCl should not, as described in U.S. Pat. No. 3,234,253, be introduced subsequently into the reaction mixture of amine and phosgene.

In the process of the present invention, mixing of the reactants takes place in a mixing apparatus in which high shear is applied to the reaction stream passed through the mixing apparatus. Preferred mixing apparatuses are rotary mixing apparatuses, mixing pumps and mixing nozzles installed upstream of the reactor. Particular preference is given to using a mixing nozzle. The mixing time in this mixing apparatus is usually from 0.0001 s to 5 s, preferably from 0.0005 to 4 s, particularly preferably from 0.001 s to 3 s. For the present purposes, the mixing time is the time taken from commencement of the mixing process for 97.5% of the fluid elements of the mixture obtained to have a mixing fraction which deviates by less than 2.5% from the theoretical final value of the mixing fraction of the mixture obtained when a state of perfect mixing has been reached. (With regard to the concept of the mixing fraction, cf., for example, J. Warnatz, U. Maas, R. W. Dibble: Verbrennung, Springer Verlag, Berlin Heidelberg New York, 1997, 2nd edition, p. 134).

In a preferred embodiment, the reaction of amine with phosgene is carried out at absolute pressures of from 0.9 bar to 400 bar, preferably from 1 to 200 bar, particularly preferably from 1.1 to 100 bar, very particularly preferably from 1.5 to 40 bar and in particular from 2 to 20 bar. The molar ratio of phosgene to amine groups used is generally from 1.1:1 to 12:1, preferably from 1.25:1 to 10:1, particularly preferably from 1.5:1 to 8:1 and very particularly preferably from 2:1 to 6:1. The total residence time in the reactors is generally from 10 seconds to 15 hours, preferably from 3 minutes to 12 hours. The reaction temperature is generally from 25 to 260° C., preferably from 35 to 240° C.

The process of the present invention is suitable for preparing all customary aliphatic and aromatic isocyanates, or a mixture of two or more such isocyanates. Preference is given to, for example, monomeric methylenedi(phenyl isocyanate) (m-MDI) or polymeric methylenedi(phenyl isocyanate) (p-MDI), tolylene diisocyanate (TDI), R,S-1-phenylethyl isocyanate, 1-methyl-3-phenylpropyl isocyanate, naphthyl diisocyanate (NDI), n-pentyl isocyanate, 6-methyl-2-heptane isocyanate, cyclopentyl isocyanate, hexamethylene diisocyanate (HDI), isophorone diisocyanate (IPDI), diisocyanatomethylcyclohexane (H₆TDI), xylylene diisocyanate (XDI), diisocyanatocyclohexane (t-CHDI), di(isocyanatocyclohexyl)methane (H₁₂MDI).

The process is particularly preferably used for preparing TDI, m-MDI, p-MDI, HDI, IPDI, H6TDI, H12MDI, XDI, t-CHDI and NDI, in particular for preparing TDI, m-MDI, p-MDI.

The process of the present invention encompasses continuous, semicontinuous and batch processes. Preference is given to continuous processes.

The isocyanates are usually prepared by reacting the corresponding primary amine with an excess of phosgene. This process preferably takes place in the liquid phase.

An additional inert solvent can be added in the process of the present invention. This additional inert solvent is usually an organic solvent or a mixture thereof. Preference is given to chlorobenzene, dichlorobenzene, trichlorobenzene, toluene, hexane, diethyl isophthalate (DEIP), tetrahydrofuran (THF), dimethylformamide (DMF), benzene and mixtures thereof. The isocyanate prepared in the plant can also be used as solvent. Particular preference is given to chlorobenzene and dichlorobenzene, and also toluene.

The amine content of the amine/solvent mixture is usually in the range from 1 to 50% by mass, preferably from 2 to 40% by mass, particularly preferably from 3 to 30% by mass.

After the reaction, the reaction mixture is preferably separated into isocyanate(s), solvent, phosgene and hydrogen chloride by means of rectification. Small amounts of by-products remaining in the isocyanate(s) can be separated off from the desired isocyanate(s) by means of additional rectification or else crystallization.

Depending on the reaction conditions chosen, the product may further comprise inert solvent, carbamoyl chloride and/or phosgene and can be processed further by known methods.

After the reaction is complete, the hydrogen chloride formed and the excess phosgene are usually separated off from the reaction mixture by distillation or by stripping with an inert gas. The hydrogen chloride/phosgene mixture is usually separated into hydrogen chloride and phosgene by distillation (FR 1 469 105) or by scrubbing with a hydrocarbon, with the outlay required for the separation of HCl and phosgene being determined by the purity requirements for the HCl and the phosgene. Here, a distinction is made between the phosgene content of the HCl and the HCl content of the phosgene. The resulting phosgene which has been freed of HCl is mixed with fresh phosgene from the phosgene synthesis and fed back to the reaction for preparing the isocyanate.

Depending on the mode of operation of the plant, the phosgene-containing stream which is fed to the reaction or mixing apparatus comprises not only phosgene and the abovementioned proportions of HCl but also the solvent in which the phosgenation is carried out. This is particularly the case when the separation of the phosgene and the hydrogen chloride is carried out by scrubbing with the solvent.

The amount of HCl present in the phosgene according to the present invention can be adjusted by recombining at least part of the HCl stream which has been separated off with the phosgene stream, or by reducing the purity requirements for the phosgene stream in respect of the specification for the HCl content. The HCl-containing phosgene stream is preferably achieved by means of a low specification and purification of the phosgene stream. For example, FR 1 469 105 describes the separation of HCl and phosgene by distillation. This is usually achieved by feeding the mixture comprising HCl and phosgene into a distillation column at a point between the stripping section and the enrichment section. The task according to the present invention is then the fractionation of the mixture comprising HCl and phosgene in a purely enrichment operation without a stripping section, with the gas stream comprising HCl and phosgene being fed into the bottom of the column. A further embodiment according to the present invention is the use of a column for the separation of mixtures comprising HCl and phosgene, with the enrichment section having at least twice as many theoretical plates as the stripping section, preferably at least three times as many theoretical plates as the stripping section and very particularly preferably at least four times as many theoretical plates as the stripping section. According to the present invention, the fractionation of the mixture comprising HCl and phosgene can be aided by the runback in the enrichment section comprising solvent. For this purpose, preference is given to introducing a solvent stream at the top of the HCl/phosgene separation.

At the same time, the omission according to the present invention of a high-efficiency separation of HCl and phosgene reduces the phosgene holdup in the plant, since the primarily phosgene-containing stripping section of the column for HCl/phosgene separation is dispensed with.

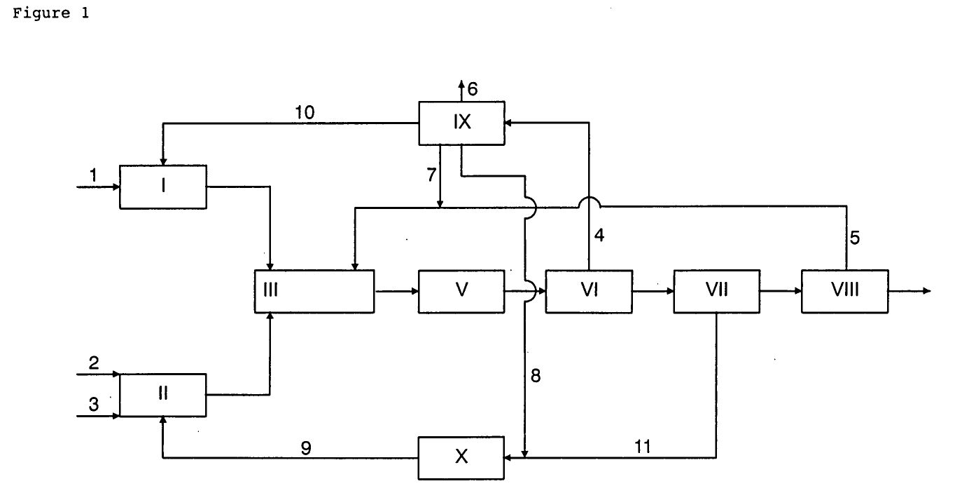

The present invention further provides a production plant which is suitable for carrying out the process of the present invention. A preferred embodiment of a production plant according to the present invention is illustrated by a general process scheme as shown in FIG. 1. Items shown in FIG. 1 are as follows:

I Phosgene reservoir
II Amine reservoir
III Mixing apparatus
V Reactor
VI First work-up apparatus
VII Second work-up apparatus
VIII Isocyanate receiver
IX Phosgene work-up
X Solvent work-up
1 Introduction of phosgene-containing feed stream
2 Introduction of amine-containing feed stream
3 Introduction of inert solvent
4 Hydrogen chloride, phosgene, inert solvent and small amounts of isocyanate which have been separated off
5 Recirculated isocyanate stream (optional)
6 Discharged hydrogen chloride
7 Isocyanate which has been separated off
8, 11 Inert solvent which has been separated off
9 Worked up inert solvent
10 Worked up phosgene

The amine from the amine reservoir II and phosgene from the phosgene reservoir I are mixed in a suitable mixing apparatus III. In an optional embodiment, the mixture of amine and phosgene is additionally mixed with recirculated isocyanate as solvent. After mixing, the mixture is transferred to a reactor V. It is likewise possible to use apparatuses which serve both as mixing and reaction apparatus, for example tube reactors having flanged-on nozzles.

In the work-up apparatus VI, hydrogen chloride and possibly inert solvent and/or small amounts of the isocyanate stream are usually separated off from the isocyanate stream.

In the optional work-up apparatus VII, inert solvent is preferably separated off and subsequently worked up (X) and returned to the amine reservoir II. For example, customary distillation units can serve as work-up apparatuses.

The process of the present invention has the advantage that an increase in the yield is achieved. At the same time, the phosgene holdup in the separation of the streams comprising HCl and phosgene can be reduced by simplification of the process.

### EXAMPLE

### Phosgenation of the Free Amine Using HCl-Containing Phosgene

A solution comprising 0.16 kg of phosgene and 0.018 kg of monochlorobenzene (MCB) was placed in a stirring autoclave apparatus at a temperature of 5° C. The solution was saturated with hydrogen chloride (HCl) by passing HCl into it at 5° C. and a pressure of 8 bar. This corresponds to an HCl content in the mixture of phosgene, HCl and MCB of 11% by mass. 0.116 kg of a solution which comprised 10% by weight of 1,6-hexamethylenediamine and 90% by weight of MCB and had a temperature of 25° C. was subsequently pumped in over a period of 10 minutes while stirring. The reaction mixture was heated to 155° C. in the stirring autoclave apparatus. The pressure in the apparatus was maintained at 4.5 bar absolute by continuously introducing a phosgene/HCl gas stream containing 2% by mass of HCl at a total mass flow of 0.05 kg/h while simultaneously releasing reaction gases. A clear solution was obtained after 7 hours. After cooling and depressurization, the residual phosgene was stripped from the solution by means of nitrogen. The yield of hexamethylene diisocyanate was 92% of theory.

### COMPARATIVE EXAMPLE WITHOUT ADDITION OF HCl TO THE PHOSGENE

A solution comprising 0.16 kg of phosgene having an HCl content of 0.5% by mass and 0.018 kg of monochlorobenzene (MCB) was placed in a stirring autoclave apparatus at a temperature of 5° C. 0.116 kg of a solution which comprised 10% by weight of 1,6-hexamethylenediamine and 90% by weight of MCB and had a temperature of 25° C. was subsequently pumped in over a period of 10 minutes while stirring. The reaction mixture was heated to 155° C. in the stirring autoclave apparatus. The pressure in the apparatus was maintained at 4.5 bar absolute by continuously introducing a phosgene/HCl gas stream containing 0.5% by mass of HCl at a total mass flow of 0.05 kg/h while simultaneously releasing reaction gases. A clear solution in which scattered solid flocs were still present was obtained after 7 hours. After cooling and depressurization, the residual phosgene was stripped from the solution by means of nitrogen. The yield of hexamethylene diisocyanate was 77% of theory.

1. A process for preparing isocyanates, comprising reacting amines with phosgene, wherein the phosgene feed stream to the reaction has a hydrogen chloride content of from 1.3% to 15% by mass.

2. The process as claimed in claim 1, wherein the phosgene feed stream is mixed with an amine feed stream in a mixing time of from 0.0001 seconds to 5 seconds.

3. The process as claimed in claim 1, wherein said isocyanates are selected from the group consisting of tolylene diisocyanate, monomeric methylenedi(phenyl isocyanate), polymeric methylenedi(phenyl isocyanate), hexamethylene diisocyanate, isophorone diisocyanate (IPDI), diisocyanatomethylcyclohexane, di(isocyanatocyclohexyl)methane, xylylene diisocyanate, diisocyanatocyclohexane and naphthyl diisocyanate.

4. The process as claimed in claim 1, wherein the reaction is carried out in a temperature range from 25 to 260° C. and at absolute pressures of from 0.9 bar to 400 bar, with a molar ratio of phosgene to amino groups being from 1.1:1 to 12:1.

5. A process for preparing isocyanates by phosgenation of primary amines, which comprises reacting phosgene having a hydrogen chloride content of from 1.3% to 15% by mass with a primary amine.

6. The process as claimed in claim 5, wherein the preparation of isocyanates is carried out in a continuous process and the reaction of phosgene with the primary amine occurs in the liquid phase.

7. A production plant for preparing isocyanates by reacting primary amines with phosgene, which comprises an amine reservoir, a phosgene reservoir, a mixing apparatus, a reactor and a work-up apparatus, wherein the phosgene feed stream fed into the mixing apparatus from the phosgene reservoir has a hydrogen chloride content of from 1.3% to 15% by mass.
